(19) United States
(12) Patent Application Publication
(10) Pub. No.: US 2025/0241670 A1
(43) Pub. Date: Jul. 31, 2025

(54) AORTIC LEAFLET CROSSING SYSTEMS WITH GRASPERS

(71) Applicant: Medtronic Vascular, Inc., Santa Rosa, CA (US)

(72) Inventors: Timothy D. FARRELL, Galway (IE); Evelyn C. COLLIER, Galway (IE); Kate MOLAN, Galway (IE); David J. OTOOLE, Galway (IE)

(21) Appl. No.: 19/034,944

(22) Filed: Jan. 23, 2025

Related U.S. Application Data
(60) Provisional application No. 63/625,340, filed on Jan. 26, 2024.

(51) Int. Cl. A61B 17/29; A61B 17/00; A61B 17/30; A61F 2/24; A61M 25/09

(52) U.S. Cl. CPC A61B 17/29; A61F 2/2436; A61B 2017/00243; A61B 2017/306; A61M 25/09

(57) ABSTRACT

An aortic leaflet crossing system includes a guide catheter, a grasper, and a guidewire. The guide catheter defines a grasper lumen and a guidewire lumen therein. The guide catheter has a proximal end defining a grasper lumen proximal opening and a guidewire lumen proximal opening. The guide catheter also has a distal end defining a grasper lumen distal opening and a guidewire lumen distal opening. The grasper is configured to extend into the grasper lumen proximal opening and through the grasper lumen and out of the grasper lumen distal opening. The guidewire is configured to extend into the guidewire lumen proximal opening and through the guidewire lumen and out of the guidewire lumen distal opening. The grasper is configured to grasp one or more aortic leaflets to form an opening across an aortic crossing. The opening is configured to receive the guidewire extending out of the guidewire lumen distal opening.

### CROSS-REFERENCE TO RELATED APPLICATIONS

This application claims the benefit of U.S. Provisional Patent Application Ser. No. 63/625,340, filed Jan. 26, 2024, the entire content of which is incorporated herein by reference.

### TECHNICAL FIELD

The present application is generally related to aortic leaflet crossing systems with graspers.

### BACKGROUND

Prosthetic heart valve designs aim to mimic the function of natural heart valve designs. One type of prosthetic heart valve design is referred to as a self-expanding valve (e.g., a supra-annular design). The self-expanding valve design includes an expandable stent frame formed of a self-expanding material (e.g., a nickel titanium alloy or Nitinol). Prosthetic leaflets may be mounted onto the expandable stent frame to replicate natural aortic leaflets.

Prosthetic heart valve delivery systems are delivered using procedures which may utilize several guidewires and catheters to provide a guided path for delivery of the prosthetic heart valve to an implementation site. This guided path extends through the aortic leaflets and typically includes guidewires and catheters extending through the opening defined by the aortic leaflets. Stenosed aortic leaflets do not open as much as healthy aortic leaflets. Therefore, applying the prosthetic heart valve delivery procedure to stenosed aortic leaflets may be challenging due to the difficulty of crossing the stenosed aortic leaflets with guidewires and catheters.

### SUMMARY

In a first embodiment, an aortic leaflet crossing system including a guide catheter, a grasper, and a guidewire is disclosed. The guide catheter defines a grasper lumen and a guidewire lumen therein. The guide catheter has a proximal end defining a grasper lumen proximal opening and a guidewire lumen proximal opening. The guide catheter also has a distal end defining a grasper lumen distal opening and a guidewire lumen distal opening. The grasper is configured to extend into the grasper lumen proximal opening and through the grasper lumen and out of the grasper lumen distal opening. The guidewire is configured to extend into the guidewire lumen proximal opening and through the guidewire lumen and out of the guidewire lumen distal opening. The grasper is configured to grasp one or more aortic leaflets to form an opening across an aortic crossing. The opening is configured to receive the guidewire extending out of the guidewire lumen distal opening.

The grasper may include a first grabber having a first grabber distal portion and a second grabber having a second grabber distal portion. The first and second grabber distal portions may be configured to collectively grasp an aortic leaflet of the one or more leaflets in an extension position. The first grabber distal portion may include one or more first grabbing features and/or the second grabber distal portion may include one or more second grabbing features. The one or more first grabbing features may be formed on a first inner surface of the first grabber distal portion and/or the one or more second grabbing features may be formed on a second inner surface of the second grabber portion.

The grasper may include a distal end portion having a first portion, a second portion, and a bend connecting the first portion and the second portion. The second portion may be configured to hook into an aortic leaflet of the one or more aortic leaflets in an extension position. The first portion may bias outward a longitudinal axis of the guide catheter and the second portion mat bias inward toward the longitudinal axis in the extension position. The grasper may be configured to retract to pull on the aortic leaflet of the one or more aortic leaflets to form the opening across the aortic crossing.

The grasper may include a first grasper having a first grasper distal portion and a second grasper having a second grasper distal portion. The first and second grasper distal portions may include first and second clamping arms configured to grasp a first aortic leaflet and a second aortic leaflet, collectively, of the one or more aortic leaflets, in an extension position. The first grasper distal portion and the second grasper distal portion may form a clamp in the extension position. The first grasper distal portion may include a first hook arm hooking into the first aortic leaflet and the second grabber distal portion may include a second hook arm hooking into the second aortic leaflet in the extension position.

In a second embodiment, an aortic leaflet crossing system including a guide catheter, a grasper, and a guidewire is disclosed. The guide catheter defines a grasper lumen and a guidewire lumen therein. The guide catheter has a proximal end defining a grasper lumen proximal opening and a guidewire lumen proximal opening. The guide catheter also has a distal end defining a grasper lumen distal opening and a guidewire lumen distal opening. The grasper is configured to extend into the grasper lumen proximal opening and through the grasper lumen and out of the grasper lumen distal opening. The guidewire is configured to extend into the guidewire lumen proximal opening and through the guidewire lumen and out of the guidewire lumen distal opening. The grasper is configured to grasp one or more aortic leaflets to form an opening across an aortic crossing. The opening is configured to receive the guidewire extending out of the guidewire lumen distal opening. The guide catheter extends along a guide catheter longitudinal axis. The grasper lumen extends along a grasper lumen longitudinal axis. The guidewire lumen extends along a guidewire lumen longitudinal axis.

The grasper lumen longitudinal axis and the guidewire lumen longitudinal axis may be offset the guide catheter longitudinal axis. The guidewire lumen longitudinal axis and the guide catheter longitudinal axis may be coextensive. The grasper lumen may include a first grasper lumen having a first grasper lumen longitudinal axis and a second grasper lumen having a second grasper lumen longitudinal axis. The first grasper lumen longitudinal axis and the second grasper lumen longitudinal axis may be offset the guide catheter longitudinal axis. The first grasper lumen longitudinal axis, the second grasper lumen longitudinal axis, and the guide catheter longitudinal axis may be coextensive along a plane.

In another embodiment, an aortic leaflet crossing method is disclosed. The method includes inserting a guide catheter defining a grasper lumen and a guidewire lumen therein into a patient's vasculature. The guide catheter has a proximal end defining a grasper lumen proximal opening and a guidewire lumen proximal opening. The guide catheter has a distal end defining a grasper lumen distal opening and a guidewire lumen distal opening. The grasper lumen carries a grasper and the guidewire lumen carries a guidewire. The method further includes delivering the guide catheter to an aorta. The method also includes advancing the grasper relative to the guide catheter out of the grasper lumen distal opening. The method also includes grasping one or more aortic leaflets with the grasper and forming an opening across an aortic crossing with the grasper. The method also advances the guidewire relative to the guide catheter out of the guidewire lumen distal opening and through the opening across the aortic crossing.

The forming step may include retracting the grasper when it is grasping the one or more aortic leaflets. The grasping step may include grasping a first aortic leaflet and a second aortic leaflet of the one or more aortic leaflets to form the opening at a cusp between the first aortic leaflet and the second aortic leaflet. The grasping step may include clamping the first aortic leaflet and the second aortic leaflet between the grasper. The grasping step may include hooking the first aortic leaflet and the second aortic leaflet.

### BRIEF DESCRIPTION OF THE DRAWINGS

FIG. 1A depicts a cross-sectional view of a patient anatomy and a schematic, side view of a pre-curved guidewire extending through the patient anatomy.

FIG. 1B depicts a cross-sectional view of the patient anatomy and a schematic, side view of a delivery system tracking over the pre-curved guidewire in a pre-deployment state.

FIG. 1C depicts a cross-sectional view of the patient anatomy and a schematic, side view of a transcatheter aortic valve replacement (TAVR) device in a partially deployed position.

FIG. 1D depicts a cross-sectional view of the patient anatomy and a schematic, side view of the TAVR device in a fully deployed position.

FIG. 2A depicts a cross-sectional view of a patient anatomy and a schematic, side view of a guide catheter extending through the patient anatomy.

FIG. 2B depicts a top view of a stenosed valve in an open state.

FIG. 2C depicts a top view of a stenosed valve in a closed state.

FIG. 2D depicts a top view of a healthy valve in an open state.

FIG. 2E depicts a top view of a healthy valve in a closed state.

FIG. 3A depicts a cross-sectional view of a patient anatomy and a schematic, side view of an aortic leaflet crossing system with a grasper in a delivery position.

FIG. 3B depicts a cross-sectional view of the aortic leaflet crossing system taken along line 3B-3B of FIG. 3A.

FIGS. 3C, 3D, and 3E depict schematic, side views of aortic leaflet crossing system 302 with grasper 304 in an extended position, a retracting position, and withdrawing position, respectively.

FIG. 4A depicts a cross-sectional view of a patient anatomy and a schematic, side view of an aortic leaflet crossing system with a grasper in a delivery position.

FIG. 4B depicts a cross-sectional view of the aortic leaflet crossing system taken along line 4B-4B of FIG. 4A.

FIG. 4C depicts a schematic, side view of an aortic leaflet crossing system with the grasper in an extending position and a retracting position.

FIG. 5A depicts a cross-sectional view of a patient anatomy and a schematic, side view of an aortic leaflet crossing system.

FIG. 5B depicts a cross-sectional view of the aortic leaflet crossing system taken along line 5B-5B of FIG. 5A.

FIG. 5C depicts a cross-sectional view of the aortic leaflet crossing system taken along line 5C-5C of FIG. 5A.

FIGS. 5D and 5E depict schematic, side views of the aortic leaflet crossing system with a clamping grasper in first and second clamping positions.

FIGS. 5F and 5G depict a schematic, side view of the aortic leaflet crossing system with a hook grasper in first and second positions, respectively.

FIG. 6 depicts a cross-sectional view of an aortic crossing and a schematic, side view of an aortic leaflet crossing system including a suction tube.

FIG. 7A depicts a schematic, side perspective view of an aortic leaflet crossing system including a guide catheter and suction tubes.

FIG. 7B depicts a cross-sectional view of the guide catheter shown in FIG. 7A.

FIG. 7C depicts a cross-sectional view of an aortic crossing and a schematic, side view of the aortic leaflet crossing system shown in FIG. 7A.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

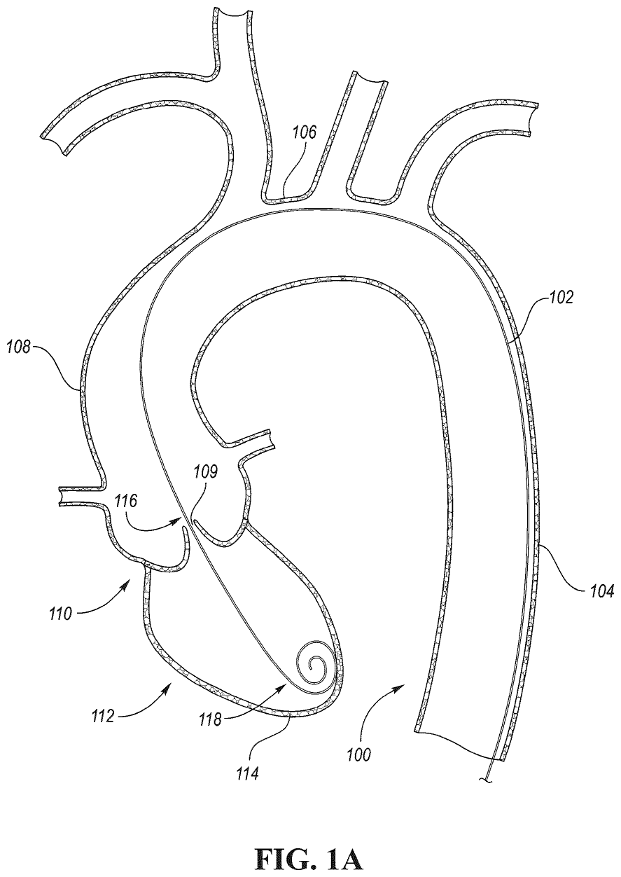

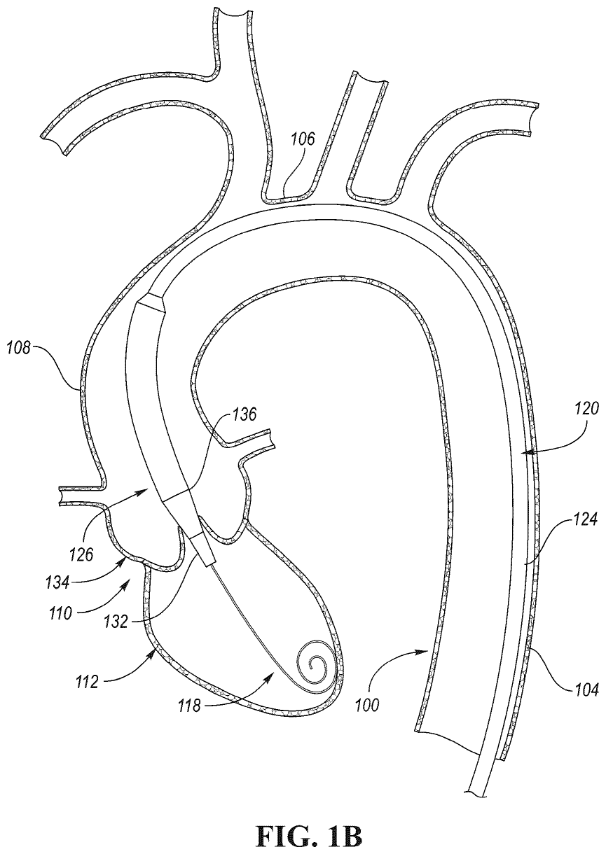

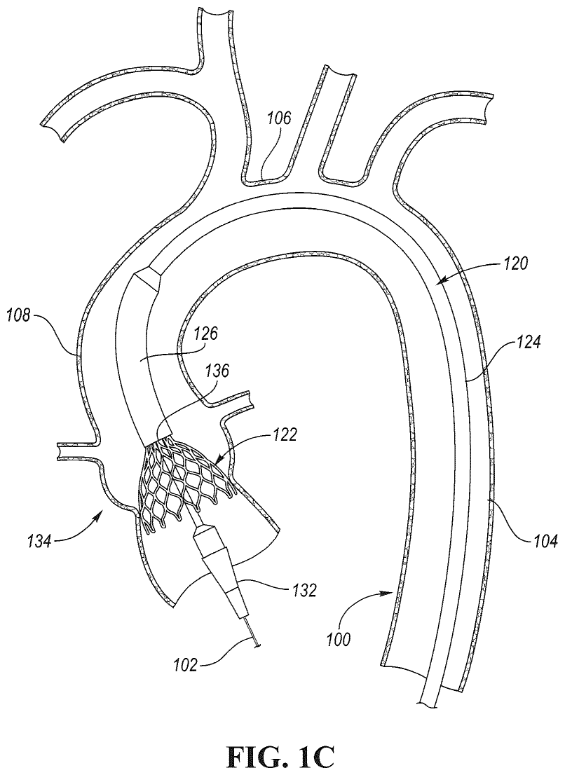

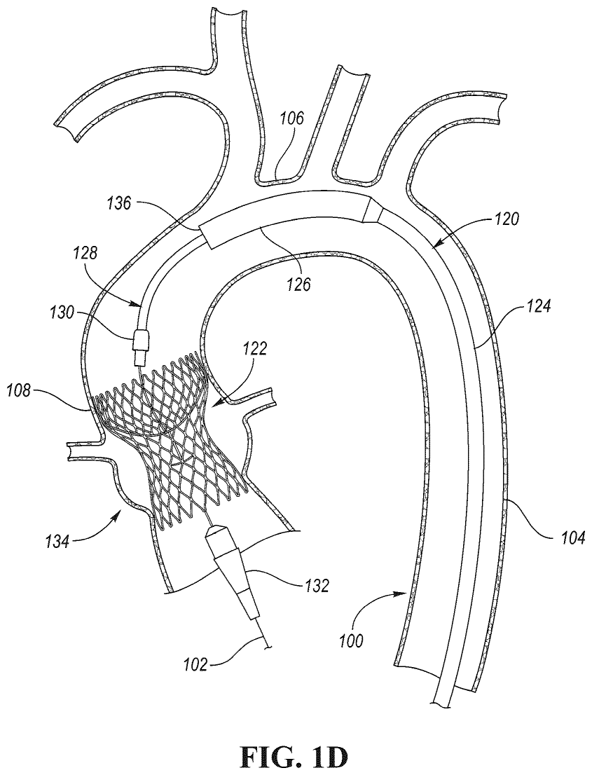

FIG. 1A depicts a cross-sectional view of patient anatomy 100 and a schematic, side view of pre-curved guidewire 102 extending through patient anatomy 100. Patient anatomy 100 includes descending aorta 104, aortic arch 106, ascending aorta 108, aortic leaflets 110, and heart 112 having heart wall 114. Descending aorta 104 is in communication with aortic arch 106. Aortic arch 106 is in communication with ascending aorta 108. Ascending aorta 108 is in communication with aortic leaflets 110 of an aortic valve.

Aortic leaflets 110 may be native aortic leaflets or replacement, prosthetic aortic leaflets. The replacement aortic leaflets may be a surgical aortic valve replacement (SAVR) device or a transcatheter aortic valve replacement (TAVR) device, such as a self-expanding TAVR device or a balloon inflatable TAVR device. Such devices generally include a frame (e.g., a stent frame) and a prosthetic valve. A TAVR device may be delivered to an implantation site using pre-curved guidewire 102 to track the TAVR device in a delivery state around patient anatomy 100 and through aortic leaflets 110.

Pre-curved guidewire 102 includes substantially straight portion 116 and pre-curved portion 118 extending from substantially straight portion 116. In one or more embodiments, pre-curve portion 118 forms a flexible tip region having greater flexibility than substantially straight portion 116, which may be considered a stiff region. Pre-curved portion may be a self-expanding structure formed of a metal alloy (e.g., stainless steel alloy, nickel titanium alloy, Nitinol, or other shape memory material).

As shown in FIG. 1A, pre-curved guidewire 102 extends through opening 109 defined by aortic leaflets 110 in a delivery position. The delivery position may be achieved using the following procedure. First, a straight tip guidewire is inserted into a patient's vasculature and advanced through opening 109 of aortic leaflets 110. Subsequently, a catheter (e.g., angiographic catheter) is placed over the straight tip guidewire and is advanced into heart 112 (e.g., a ventricle of heart 112). The straight tip guidewire is then removed from the catheter and the patient's vasculature, and replaced with a J-tip wire, which is inserted into the patient's vasculature and advanced through the catheter. The catheter may then be exchanged for a second catheter (e.g., pigtail catheter) by withdrawing the catheter and then inserting the second catheter into the patient's vasculature and advancing it over the catheter-tip wire. The J-tip wire is then exchanged for pre-curved guidewire 102 by advancement through the second catheter. While pre-curved guidewire 102 advances through the second catheter, pre-curved portion 118 is straightened by the second catheter in a constrained state. When pre-curved portion 118 emerges from the distal end of the second catheter, pre-curved portion 118 takes on its pre-curved shape as shown in FIG. 1A and may contact heart wall 114. The above process for establishing a curved guidewire in the left ventricle is merely an example, and other steps or devices may be used to establish the guidewire.

FIG. 1B depicts a cross-sectional view of patient anatomy 100 and a schematic, side view of delivery system 120 tracking over pre-curved guidewire 102 in a pre-deployment state. FIG. 1C depicts a cross-sectional view of patient anatomy 100 and a schematic, side view of delivery system 120 and TAVR device 122 in a partially deployed position. FIG. 1D depicts a cross-sectional view of patient anatomy 100 and a schematic, side view of delivery system 120 and TAVR device 122 in a fully deployed position.

Delivery system 120 includes delivery sheath assembly 124, capsule 126, inner shaft assembly 128, retention hub 130, and tip 132. Delivery system 120 is configured to retain TAVR device 122 in a delivery state in which TAVR device 122 is loaded in a constrained position within delivery system 120. In the delivery state, TAVR device 122 is coupled to inner shaft assembly 128 via retention hub 130 (e.g., TAVR device 122 may include eyelets captured by retention hub 130) and is compressively retained within capsule 126 of delivery sheath assembly 124. Loaded delivery system 120 may be configured to percutaneously deliver TAVR device 122 to implantation site 134 (e.g., a defective heart valve). As shown in FIGS. 1C and 1D, loaded delivery system 120 may be advanced toward implantation site over pre-curved guidewire 102 in a retrograde manner through the patient's femoral artery into descending aorta 104 over aortic arch 106 through ascending aorta 108 and across a defective heart valve (e.g., about mid-way through defective heart valve) at implantation site 134.

For self-expanding embodiments, delivery sheath assembly 124 is configured to withdraw capsule 126 proximally from TAVR device 122 via operation of a handle (not shown) on delivery system 120, thereby allowing TAVR device 122 to expand from the constrained, pre-deployment position to a partially deployed position shown in FIG. 1C and fully deployed position shown in FIG. 1D in which TAVR device 122 is fully released from capsule 126 and TAVR device 122 is implanted to the native valve. A release sheath assembly of delivery system 120 may be configured to fully release TAVR device 122 from capsule 126. The handle may be configured to maneuver capsule 126 to the partially deployed position shown in FIG. 1C in which a distal region of TAVR device 122 is permitted to self-expand at distal end 136 of retention hub 130 while a proximal region of TAVR device 122 remains coupled to retention hub 130.

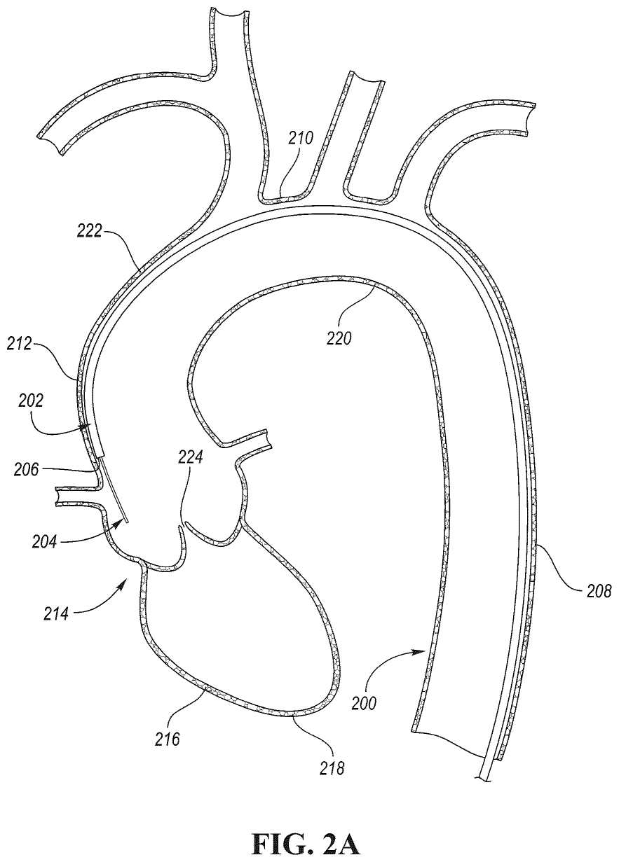

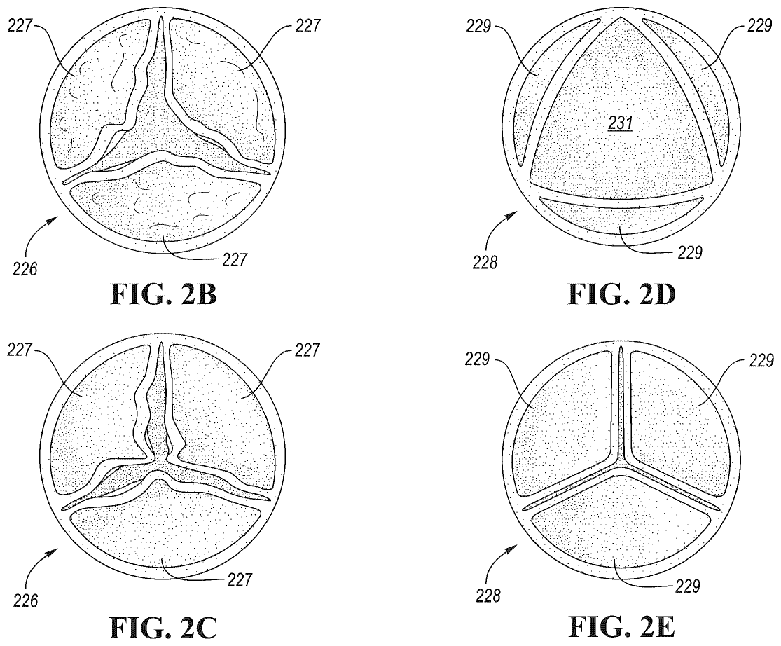

FIG. 2A depicts a cross-sectional view of patient anatomy 200 and a schematic, side view of guide catheter 202 extending through patient anatomy 200. As shown in FIG. 2A, straight tip guidewire 204 has extended through distal end 206 of guide catheter 202. Patient anatomy 200 includes descending aorta 208, aortic arch 210, ascending aorta 212, aortic leaflets 214, and heart 216 having heart wall 218. The aorta includes outer curve 222 and inner curve 220.

As shown in FIG. 2A, guide catheter 202 and straight tip guidewire 204 has aligned with outer curve 222 of the aorta. This alignment may occur due to the stiffness of the body of guide catheter 202 and straight tip guidewire 204 causing these components to bow outward toward outer curve 222. As shown in FIG. 2A, guide catheter 202 and straight tip guidewire 204 are not aligned with opening 224 defined by aortic leaflets 214. This misalignment may present a challenge to deployment of a TAVR device at an implantation site.

While misalignment may increase the amount of time taken to cross opening 224, one or more other factors may increase implantation procedure time. FIG. 2B depicts a top view of stenosed valve 226 in an open state. FIG. 2C depicts a top view of stenosed valve 226 in a closed state. Stenosed valve 226 includes stenosed aortic leaflets 227. FIG. 2D depicts a top view of healthy valve 228 in an open state. FIG. 2E depicts a top view of healthy valve 228 in a closed state. Healthy valve 228 includes healthy aortic leaflets 229. In healthy valve 228, blood flows out of heart 216 through opening 231 in the open state while heart 216 beats and closes substantially in the closed state. In the open state of healthy valve 228, opening 231 provides a large opening for crossing the annulus, although the force of blood flowing out of heart 216 works against catheter(s) and/or guidewire(s) crossing the annulus. However, crossing the annulus in the closed state with healthy valve 228 substantially closed presents difficulties. The difficulties are present in both the open and closed states of stenosed valve 226, where the stenosed aortic leaflets 227 do not fully open or close but are rather in a partially closed state. Another factor that may contribute to the difficulty in crossing the annulus is that the catheter(s) and/or guidewire(s) may get caught on the aortic leaflets. These difficulties may add to procedure time where reducing procedure time may enhance the benefit of the procedure.

Considering the foregoing, what is needed is an aortic leaflet crossing system to address one or more of the factors identified above that may contribute to increased TAVR device implantation procedure time. In one or more embodiments, aortic leaflet crossing systems are disclosed that may repeatedly and consistently cross aortic leaflets with a guidewire while reducing the instance of one or more of the challenging factors identified above.

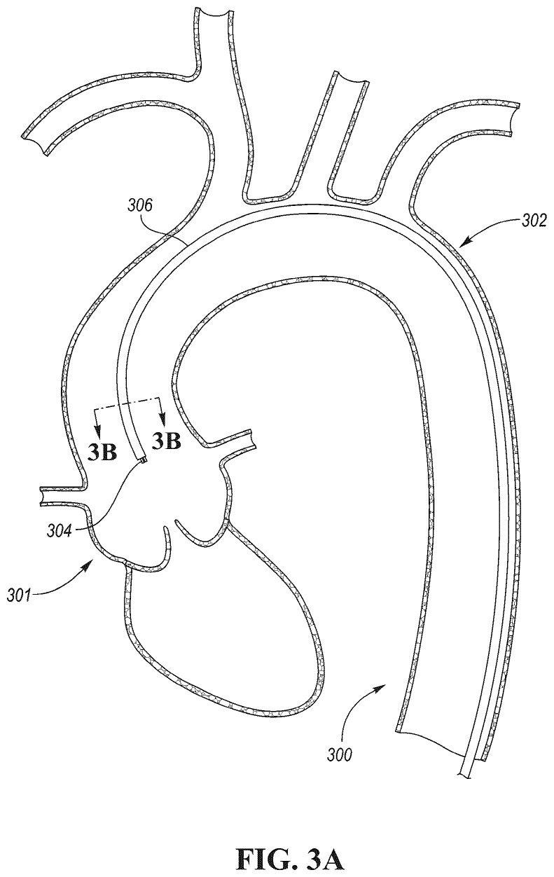

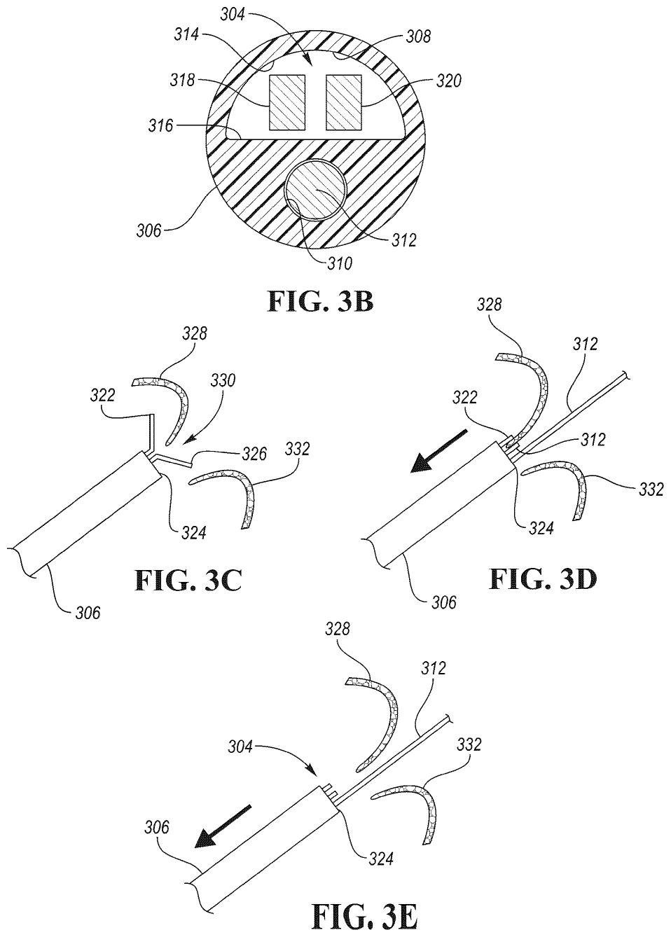

FIG. 3A depicts a cross-sectional view of patient anatomy 300 and a schematic, side view of aortic leaflet crossing system 302 with grasper 304 in a delivery position. In the delivery position, guide catheter 306, including grasper 304, enters the patient's vasculature and travels to implantation site 301. FIG. 3B depicts a cross-sectional view of aortic leaflet crossing system 302 taken along line 3B-3B of FIG. 3A. FIGS. 3C, 3D, and 3E depict schematic, side views of aortic leaflet crossing system 302 with grasper 304 in an extended position, a retracting position, and withdrawing position, respectively.

With reference to FIGS. 3A and 3B, aortic leaflet crossing system 302 includes guide catheter 306 defining first lumen 308 and second lumen 310. First lumen 308 and second lumen 310 extend along the length of guide catheter 306. Grasper 304 extends within first lumen 308. Guidewire 312 extends within second lumen 310. As shown in FIG. 3B, first lumen 308 has a half-circle cross-sectional profile including half-circle surface 314 and planar surface 316 extending between terminating ends of half-circle surface 314. In other embodiments, first lumen 308 may have other cross-sectional profiles to accommodate grasper 304. As shown in FIG. 3B, second lumen 310 has a circular cross-sectional profile. In other embodiments, second lumen 310 may have other cross-sectional profiles to accommodate guidewire 312.

Guide catheter 306 extends along a longitudinal axis. The longitudinal axis of guide catheter 306 may be coextensive with planar surface 316 of first lumen 308. Alternatively, first lumen 308 may be offset from the longitudinal axis of guide catheter 306. As shown in FIG. 3B, second lumen 310 is offset the longitudinal axis of guide catheter 306. Alternatively, a longitudinal axis of second lumen 310 may be coextensive with the longitudinal axis of guide catheter 306. In one or more embodiments, the positions of first lumen 308 and second lumen 310 within guide catheter 306 are fixed relative to each other such that the openings formed at distal end 324 of guide catheter 306 are a fixed distance from each other. The fixed distance is configured to permit alignment of grasper 304 to perform the aortic opening function and alignment of guidewire 312 to extend through the opening.

Grasper 304 includes first grabber 318 and second grabber 320. First grabber 318 includes first grabber distal portion 322. Second grabber 320 includes second grabber distal portion 326. First grabber distal portion 322 and/or second grabber distal portion 326 may extend beyond distal end 324 a relatively short distance in the delivery position shown in FIG. 3A. Alternatively, first grabber distal portion 322 and/or second grabber distal portion 326 may be contained within first lumen 308 in the delivery position. First grabber distal portion 322 and second grabber distal portion 326 may be formed of a metal material configured to bias outward as they extend from distal end 324 of guide catheter 306. The metal material may be a shape setting material to form the outwardly biasing portion of the grabber distal portions. The shape setting material may be Nitinol or a shape setting stainless steel.

Once distal end 324 of guide catheter 306 reaches implantation site 301, grasper 304 is deployed into the extended position. As shown in FIG. 3C, first grabber distal portion 322 and second grabber distal portion 326 have extended from their delivery positions into extended positions. As shown in FIG. 3C, first grabber distal portion 322 and second grabber distal portion 326 extend axially beyond distal end 324 and extend radially beyond the diameter of guide catheter 306 to create an extension gap between first grabber distal portion 322 and second grabber distal portion 326. In the extended position, first grabber distal portion 322 and second grabber distal portion 326 may be outwardly angled relative to the longitudinal axis of guide catheter 306 to form the extension gap therebetween.

First grabber 318 and second grabber 320 may be configured to extend independent of guide catheter 306 (e.g., first grabber 318 and second grabber 320 may extend while guide catheter 306 remains stationary). First grabber 318 and second grabber 320 may be extended into the extended position using an actuator. The actuator may be a slider connected to or part of the proximal ends of first grabber 318 and second grabber 320. The slider may be translated in a distal direction to actuate the extension of first grabber 318 and second grabber 320 relative to guide catheter 306. The actuator may be disposed in a handle (not shown) of aortic leaflet crossing system 302.

As shown in FIG. 3C, a portion of first leaflet 328 is located within the extension gap between first grabber distal portion 322 and second grabber distal portion 326. Second grabber distal portion 326 extends within opening 330 formed between first leaflet 328 and second leaflet 332. In the extended position shown in FIG. 3C, grasper 304 is configured to move into the retracting position as shown in FIG. 3D.

As shown in FIG. 3D, first grabber distal portion 322 and second grabber distal portion 326 have been retracted from the extension position into the retracted position. First grabber distal portion 322 and second grabber distal portion 326 may be retracted by retracting first grabber 318 and second grabber 320 using an actuator, which may be the same actuator used to extend first grabber 318 and second grabber 320 into the extended position. The actuator may be a slider connected to or part of the proximal ends of first grabber 318 and second grabber 320. The slider may be translated in a proximal direction to actuate the retraction of first grabber distal portion 322 and second grabber distal portion 326 relative to guide catheter 306. In the retracted position, first grabber distal portion 322 and second grabber distal portion 326 contact a distal end portion of first leaflet 328, thereby exerting a grasping force on the distal end portion of first leaflet 328. Alternatively, first grabber distal portion 322 and second grabber distal portion 326 may be biased closed and the actuator may be configured to maintain first grabber distal portion 322 and second grabber distal portion 326 in an open position until released so that first grabber distal portion 322 and second grabber distal portion 326 close around first leaflet 328.

After generating the grasping force, first grabber 318 and second grabber 320 may be further retracted to create a pulling force on first leaflet 328 to increase the size of opening 330. This further retraction may be generated by retracting first grabber 318 and second grabber 320 relative to guide catheter 306 or by retracting guide catheter 306 when first grabber 318 and second grabber 320 are connected to guide catheter 306. After increasing the size of opening 330, guidewire 312 is advanced through second lumen 310 at distal end 324 and through opening 330 such that guidewire 312 crosses the aortic leaflets. Second lumen 310 may be offset the longitudinal axis of guide catheter 306 and first lumen 308 such that second lumen 310, and therefore, guidewire 312, are aligned with opening 330.

In one or more embodiments, first grabber distal portion 322 and second grabber distal portion 326 may include one or more grabbing features to contact an aortic leaflet during retraction. The one or more grabbing features may be formed on the inner surfaces of first grabber distal portion 322 and/or second grabber distal portion 326. The one or more grabbing features may be one or more barbs, projections, prongs, spurs, and/or points or a high-friction material attached to the inner surfaces (e.g., a higher coefficient of friction than the grabbers).

After guidewire 312 has crossed the aortic leaflets, aortic leaflet crossing system 302 may be withdrawn from the patient's vasculature. Grasper 304 may be disengaged from the distal end of first leaflet 328 by extending first grabber distal portion 322 and second grabber distal portion 326 as disclosed herein (e.g., as shown in FIG. 3C). Once grasper 304 is disengaged from first leaflet 328, guide catheter 306 may be partially axially withdrawn in a proximal direction so that the extended first grabber distal portion 322 and second grabber distal portion 326 clear the distal end of first leaflet 328 so that first grabber distal portion 322 and second grabber distal portion 326 may be retracted without re-engaging the distal end of first leaflet 328 as shown in FIG. 3E. Aortic leaflet crossing system 302, except guidewire 312, may be withdrawn from the vasculature of the patient.

FIGS. 3A through 3B depicts 2 grabbers. In other embodiments, the number of grabbers may be greater than two (e.g., 3, 4, 5, 6, 7, 8, 9, 10, 11, or 12, or any range between two of these values). The grabbers may be located an equal radial angle from each other (e.g., 180 degrees for 2 grabbers or 90 degrees for 4 grabbers), or may be different radial angles from each other.

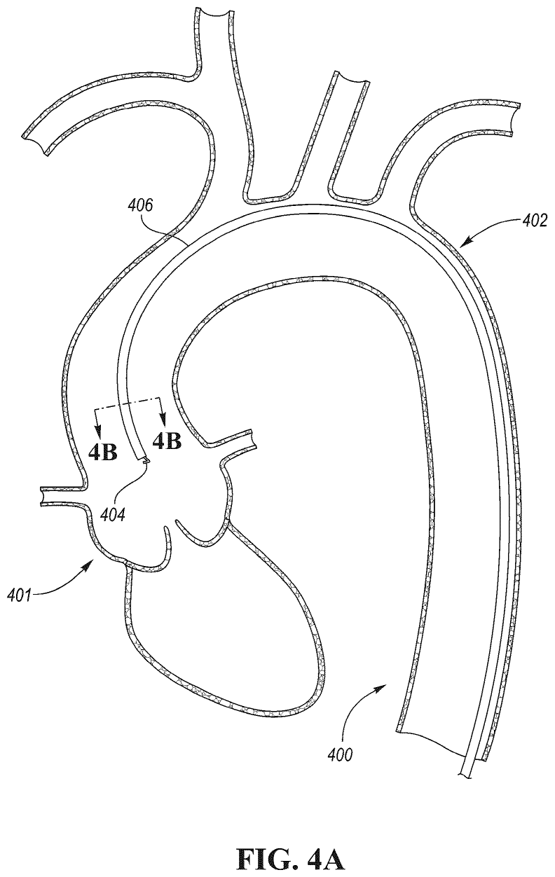

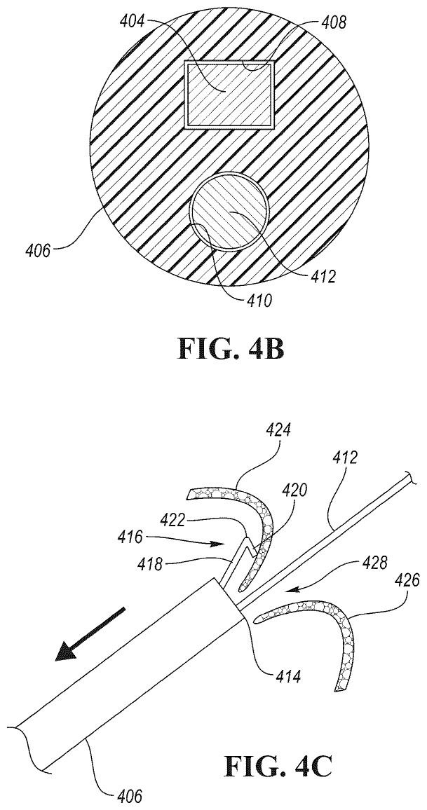

FIG. 4A depicts a cross-sectional view of patient anatomy 400 and a schematic, side view of aortic leaflet crossing system 402 with grasper 404 in a delivery position. FIG. 4B depicts a cross-sectional view of aortic leaflet crossing system 402 taken along line 4B-4B of FIG. 4A. FIG. 4C depicts a schematic, side view of aortic leaflet crossing system 402 with grasper 404 in an extending position and a retracting position.

With reference to FIGS. 4A and 4B, aortic leaflet crossing system 402 includes guide catheter 406 defining first lumen 408 and second lumen 410. First lumen 408 and second lumen 410 extends along the length of guide catheter 406. Grasper 404 extends within first lumen 408. Guidewire 412 extends within second lumen 410. As shown in FIG. 4B, first lumen 408 has a rectangular cross-sectional profile including planar side surfaces. In other embodiments, first lumen 408 may have other cross-sectional profiles to accommodate grasper 404. As shown in FIG. 3B, second lumen 410 has a circular cross-sectional profile. In other embodiments, second lumen 410 may have other cross-sectional profiles to accommodate guidewire 412.

Guide catheter 406 extends along a longitudinal axis. As shown in FIG. 4B, first lumen 408 and second lumen 410 are offset the longitudinal axis of guide catheter 406. Alternatively, first lumen 408 or second lumen 410 may be coextensive with the longitudinal axis of guide catheter 406. In one or more embodiments, the positions of first lumen 408 and second lumen 410 within guide catheter 406 are fixed relative to each other such that the openings formed at distal end 414 of guide catheter 406 are a fixed distance from each other. The fixed distance is configured to permit alignment of grasper 404 to perform the aortic leaflet opening function as described herein and alignment of guidewire 412 to extend through the opening.

Grasper 404 includes distal end portion 416 having first portion 418 and second portion 420. Bend 422 connects first portion 418 and second portion 420. Grasper 404 may be loaded into first lumen 408 with bend 422 as the leading edge. Bend 422 is configured to permit second portion 420 to collapse within first lumen 408 to conform to the size of first lumen 408. As distal end portion 416 exits first lumen 408 at distal end 414 of guide catheter 406, second portion 420 springs inwardly (e.g., as shown in FIG. 4C). Distal end portion 416 may be formed of a metal material (e.g., Nitinol, stainless steel, or other shape setting material) that is configured to permit first portion 418 to bias outwardly and second portion 420 to bias inwardly upon extending from distal end 414 of guide catheter 406.

Once distal end 414 of guide catheter 406 reaches implantation site 401 in the delivery position, grasper 404 is configured to extend into an extended position. In the extended position, first portion 418 of distal end portion 416 extends from distal end 414 of guide catheter 406 and second portion 420 extends radially inward from first portion 418 toward the longitudinal axis of guide catheter 306. The radial inward direction of second portion 420 is configured to permit distal end portion 416 to hook into leaflet 424.

Grasper 404 may be configured to extend while guide catheter 406 remains stationary. Distal end portion 416 may be extended into the extended position using an actuator (e.g., a slider connected to or part of the proximal end of grasper 404). The slider may be translated in a distal direction to actuate the extension of grasper 404 relative to guide catheter 406. The slider or other actuator may be disposed in a handle (not shown) of aortic leaflet crossing system 402.

Once second portion 420 hooks into leaflet 424, grasper 404 may be retracted using the actuator (e.g., the same slider used to extend grasper 404). The slider may be translated in a proximal direction to actuate the retraction of grasper 404 relative to guide catheter 306. In the retracted position, grasper 404 creates a pulling force on leaflet 424 to increase the size of opening 428 situated between leaflet 424 and leaflet 426. After increasing the size of opening 428, guidewire 412 may be advanced through second lumen 410 at distal end 414 and opening 428 such that guidewire 412 crosses the aortic leaflets. Second lumen 410 may be offset the longitudinal axis of guide catheter 406 and first lumen 408 such that second lumen 410, and therefore, guidewire 412, are aligned with opening 428.

After guidewire 412 crosses the aortic leaflets, aortic leaflet crossing system 402 may be withdrawn from the patient's vasculature. Grasper 404 may be disengaged from leaflet 424 by advancing and/or radially moving distal end 414 to unhook distal end 414 from leaflet 424. Once grasper 404 is disengaged from leaflet 424, aortic leaflet crossing system 402, except guidewire 412, may be withdrawn from the vasculature of the patient.

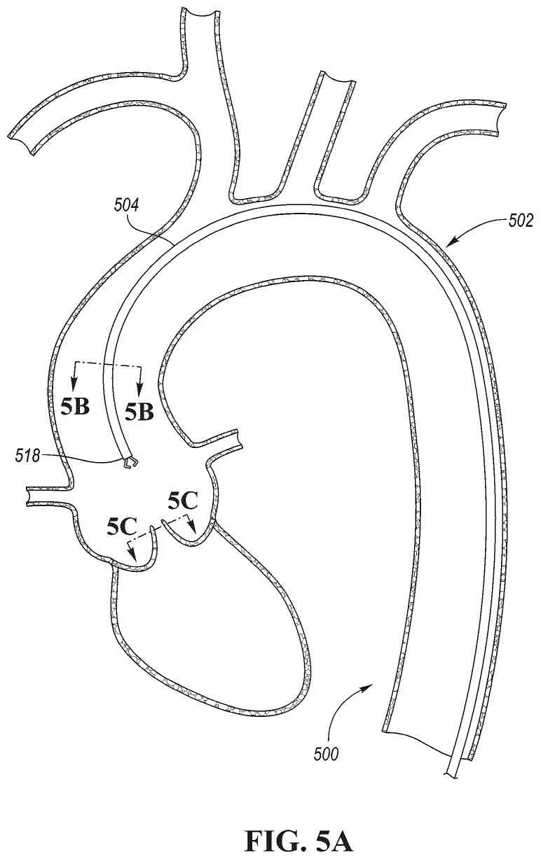

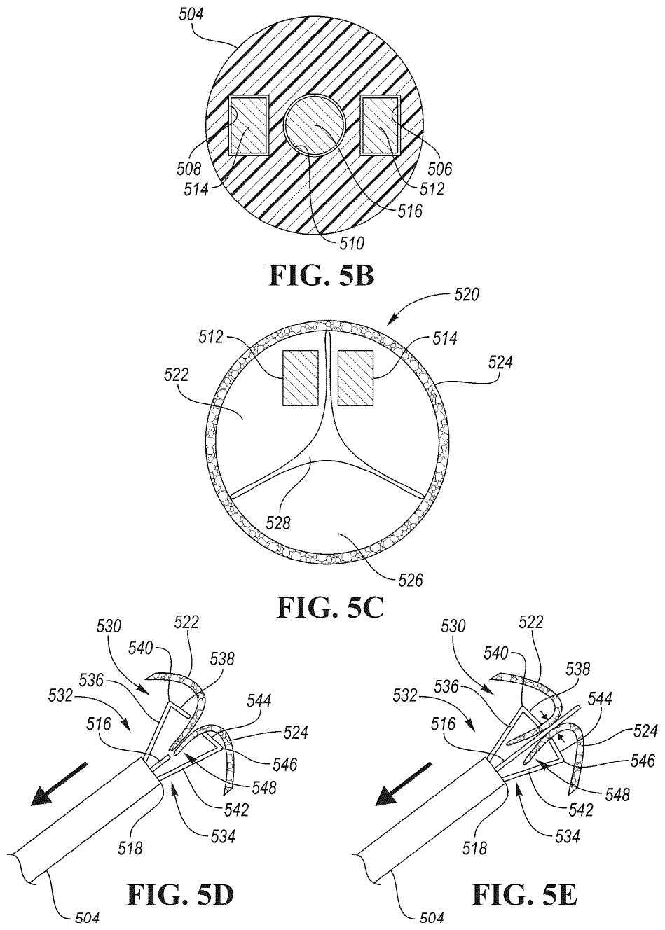

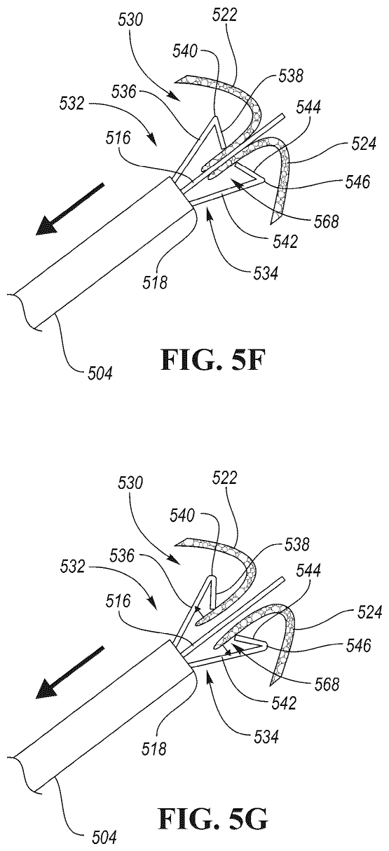

FIG. 5A depicts a cross-sectional view of patient anatomy 500 and a schematic, side view of aortic leaflet crossing system 502. FIG. 5B depicts a cross-sectional view of aortic leaflet crossing system 502 taken along line 5B-5B of FIG. 5A. FIG. 5C depicts a cross-sectional view of aortic leaflet crossing system 502 taken along line 5C-5C of FIG. 5A.

With reference to FIGS. 5A and 5B, aortic leaflet crossing system 502 includes guide catheter 504 defining first lumen 506, second lumen 508, and third lumen 510 extending along the length of guide catheter 504. First grasper 512 and second grasper 514 extend within first lumen 506 and second lumen 508, respectively. As shown in FIG. 5B, first lumen 506 and second lumen 508 have rectangular cross-sectional profiles including planar side surface. In other embodiments, first lumen 506 and second lumen 508 may have other cross-sectional profiles to accommodate first grasper 512 and second grasper 514. As shown in FIG. 5B, third lumen 510 has a circular cross-sectional profile. In other embodiments, third lumen 510 may have other cross-sectional profiles to accommodate guidewire 516.

Guide catheter 504 extends along a longitudinal axis. The longitudinal axis of guide catheter 504 may be coextensive with the longitudinal axis of third lumen 510. Alternatively, the longitudinal axis of guide catheter 504 may be offset the longitudinal axis of third lumen 510. As shown in FIG. 5B, first lumen 506 and second lumen 508 are offset from the longitudinal axis of guide catheter 504. First lumen 506 is located in a first longitudinal half of guide catheter 504 and second lumen 508 is located in a second longitudinal half of guide catheter 504. In one or more embodiments, the positions of first lumen 506 and second lumen 508 within guide catheter 504 are fixed relative to third lumen 510 such that the openings formed at distal end 518 of guide catheter 504 are first and second fixed distances, respectively, from third lumen 510. The first and second fixed distances are configured to permit alignment of first grasper 512 and second grasper 514 to perform the aortic opening function and alignment of guidewire 516 through the opening as further described below. In one or more embodiments, the first and second fixed distances may be the same or substantially the same.

With reference to FIG. 5C, aortic crossing 520 includes first leaflet 522, second leaflet 524, and third leaflet 526, collectively forming opening 528 therebetween. First leaflet 522 and second leaflet 524 are adjacent each other. Second leaflet 524 and third leaflet 526 are adjacent each other. Third leaflet 526 and first leaflet 522 are adjacent each other. First grasper 512 and second grasper 514 are configured to grasp first leaflet 522 and second leaflet 524, respectively (or any two adjacent pairs of leaflets), while not occluding the flow of blood into and out of the heart through aortic crossing 520.

FIGS. 5D and 5E depict schematic, side views of aortic leaflet crossing system 502 with guide catheter 504 and grasper 530 in first and second positions, respectively. Grasper 530 includes first arm 532 and second arm 534. First arm 532 includes first base portion 536 and first grasping portion 538 connected to each other through first bend 540. Second arm 534 includes second base portion 542 and second grasping portion 544 connected to each other through second bend 546. First bend 540 and second bend 546 are configured to permit first arm 532 and second arm 534, respectively, to collapse within first lumen 506 and second lumen 508, respectively, to conform to the size of first lumen 506 and second lumen 508, respectively.

As first arm 532 and second arm 534 respectively exit first lumen 506 and second lumen 508, first base portion 536 and second base portion 542 bias outward and first portion 538 and second portion 542 spring closed on first leaflet 522 and second leaflet 524 near the cusp therebetween. Aortic leaflet crossing system 502 may include an actuator configured to maintain first portion 538 and second portion 542 in an open position until released so that first portion 538 and second portion 542 close (e.g., first portion 538 and second portion 542 are biased closed). Alternatively, first portion 538 and second portion 542 may be biased in an open position and the actuator may be used to bring them together. This spring action is configured to lever open the cusp to create opening 548 between first leaflet 522 and second leaflet 524. As shown in FIG. 5E, guidewire 516 advances through third lumen 510, thereby centering guidewire 516 with opening 548. By clamping first leaflet 522 and second leaflet 524 relative to guide catheter 504, the clinician creates leverage to push guidewire 516 through opening 548.

FIGS. 5F and 5G depict schematic, side views of aortic leaflet crossing system 502 with guide catheter 504 and grasper 530 in first and second positions, respectively. As shown in these figures and described below, this approach grasps the aortic leaflets closer to their free ends. Grasper 530 include first arm 532 and second arm 534. First arm 532 includes first base portion 536 and first grasping portion 538 connected to each other through first bend 540. Second arm 534 includes second base portion 542 and second grasping portion 544 connected to each other through second bend 546. First bend 540 and second bend 546 are configured to permit first arm 532 and second arm 534, respectively, to collapse within first lumen 506 and second lumen 508, respectively, to conform to the size of first lumen 506 and second lumen 508, respectively.

As first arm 532 and second arm 534 respectively exit first lumen 506 and second lumen 508, first base portion 536 and second base portion 542 bias outward and first grasping portion 538 and second grasping portion 544 bias inward toward the longitudinal axis of guide catheter 504 to hook into first leaflet 522 and second leaflet 524, respectively. Once first grasping portion 538 and second grasping portion 544 hook into first leaflet 522 and second leaflet 524, respectively, then grasper 530 may be pulled back to create opening 568 between first leaflet 522 and second leaflet 524. As shown in FIG. 5E, guidewire 516 advances through third lumen 510, thereby centering guidewire 516 with opening 568.

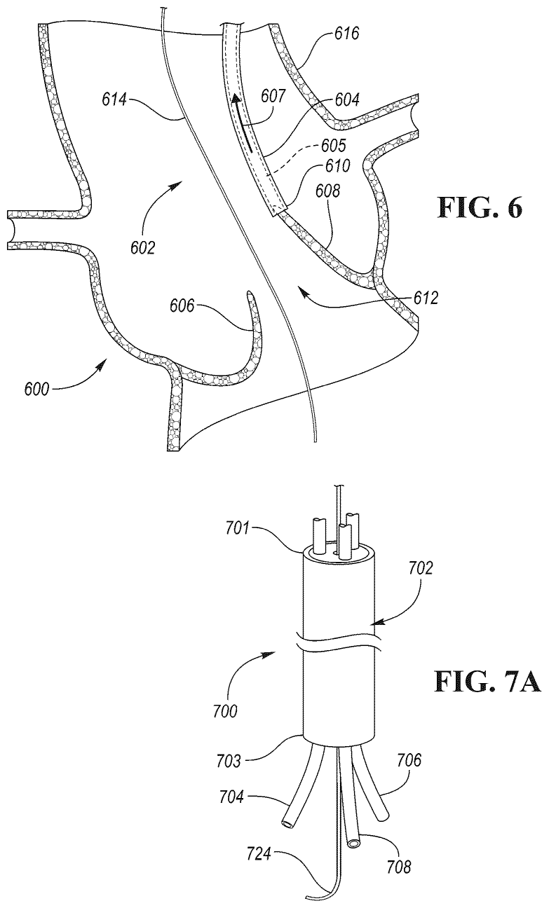

FIG. 6 depicts a cross-sectional view of aortic crossing 600 and a schematic, side view of aortic leaflet crossing system 602 including suction tube 604 defining suction lumen 605. Aortic crossing 600 includes first leaflet 606 and second leaflet 608. Suction tube 604 is inserted into a patient's vasculature and advanced to aortic crossing 600. Suction tube 604 includes a proximal end (not shown) and distal end 610. Suction tube 604 may be advanced such that distal end 610 is in the vicinity of one of the leaflets (e.g., second leaflet 608). The pressure within suction tube 604 may be decreased relative to the pressure in aortic crossing 600 such that a suction force is applied in the direction of arrow 607 to draw second leaflet 608 toward and/or in contact with distal end 610 of suction tube 604. The suction force is configured to increase the size of opening 612 between the leaflets of aortic crossing 600, thereby creating space for advancement of guidewire 614 through opening 612. Leaflet 608 is pulled out of the way by the suction force in the direction of aortic wall 616. A pump may be connected to suction tube 604 to create the suction force by generating a partial vacuum within suction lumen 605. In one embodiment, guidewire 614 may be advanced on its own or through a separate guide catheter (not shown). In another embodiment, guidewire 614 may be advanced out of a second lumen attached to or forming a part of suction tube 604. For example, a second lumen may have a distal end proximal to distal end 610 and angled relative to the longitudinal axis of suction tube 604 such that the guidewire extends at an angle relative thereto and may extend through opening 612 when leaflet 608 is pulled away by the suction.

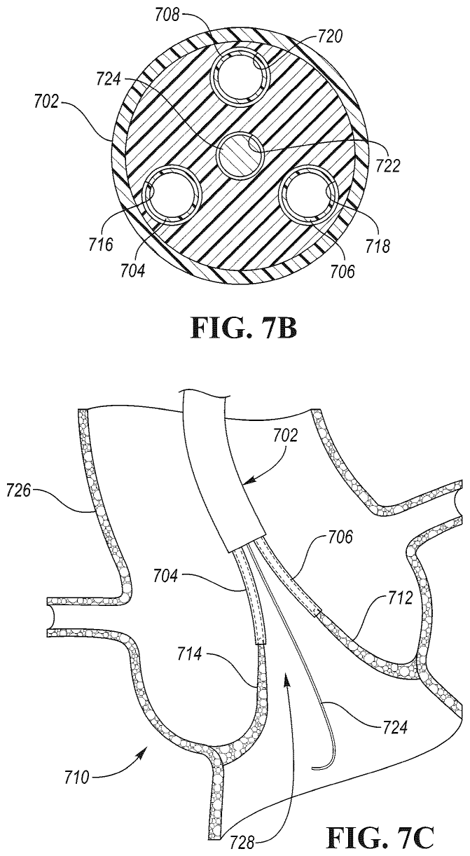

FIG. 7A depicts a schematic, side perspective view of aortic leaflet crossing system 700 including guide catheter 702 and first suction tube 704, second suction tube 706, and third suction tube 708 extending within guide catheter 702. FIG. 7B depicts a plan view of guide catheter 702 depicting proximal end 701 of guide catheter 702. FIG. 7C depicts a cross-sectional view of aortic crossing 710 and a schematic, side view of aortic leaflet crossing system 700. Aortic crossing 710 includes first leaflet 712, second leaflet 714, and third leaflet (not shown).

Guide catheter 702 defines first lumen 716, second lumen 718, third lumen 720, and fourth lumen 722. First suction tube 704 is configured to extend through first lumen 716. Second suction tube 706 is configured to extend through second lumen 718. Third suction tube 708 is configured to extend through third lumen 720. Guidewire 724 is configured to extend through fourth lumen 722.

Guide catheter 702 extends along a longitudinal axis. The longitudinal axis of guide catheter 702 may be coextensive with the longitudinal axis of fourth lumen 722. As shown in FIG. 7B, first lumen 716, second lumen 718, and third lumen 720 are offset from the longitudinal axis of guide catheter 702. As shown in FIG. 7C, first lumen 716, second lumen 718, and third lumen 720 are located an equal radial angle of 120 degrees from each other (the lumens may be different radial angles from each other in other embodiments). First lumen 716, second lumen 718, and third lumen 720 are positioned at 12 o'clock, 4 o'clock, and 8'clock. While FIGS. 7A, 7B, and 7C depict 3 suction tube/lumen pairs, in other embodiments, the number of suction tube/lumen pairs may be 2, 4, 5, or 6, or any range between two of these numbers. Using additional suction tubes may enhance the likelihood of the suction tubes contacting one or more of the aortic leaflets.

Guide catheter 702 including first suction tube 704, second suction tube 706, and third suction tube 708 extending within first lumen 716, second lumen 718, and third lumen 720, respectively, is inserted into a patient's vasculature and advanced to aortic crossing 710. During this delivery step, first suction tube 704, second suction tube 706, and third suction tube 708 may be retracted within first lumen 716, second lumen 718, and third lumen 720, respectively, such that first suction tube 704, second suction tube 706, and third suction tube 708 do not extend beyond distal end 703 of guide catheter 702. Guide catheter 702 may be advanced such that distal end 703 of guide catheter 702 is in the vicinity of one or more of the leaflets (e.g., first leaflet 712 and second leaflet 714).

Once distal end 703 of guide catheter 702 is in position, guide catheter 702 may be retracted such that first suction tube 704, second suction tube 706, and third suction tube 708 spring out into a tripod configuration as shown in FIGS. 7A and 7C where first suction tube 704, second suction tube 706, and third suction tube 708 extend beyond the outer diameter of guide catheter 702. Alternatively, suction tubes 704, 706, and 708 may be extended distally relative to guide catheter 702 to assume a tripod configuration. The tripod configuration may be accomplished via any suitable mechanism, such as a shape memory material (e.g., Nitinol) incorporated into the suction tubes, an angle formed in the distal end of the lumens 716, 718, and 720, deflecting portions extending from the distal end 703, or others. Suction is then applied to one or more of first suction tube 704, second suction tube 706, and third suction tube 708, causing one or more of first leaflet 712, second leaflet 714, and third leaflet (not shown) to be drawn towards and/or attach to one or more of first suction tube 704, second suction tube 706, and third suction tube 708, respectively. The one or more of the leaflets may be moved toward aortic wall 726 to move them out of the way to create an increase size of opening 728 between the leaflets of aortic crossing 710. Guidewire 724 is advanced through fourth lumen 720 defined by guide catheter 702. In one or more embodiments, the tripod configuration ensures that guidewire 724 is centered to pass through opening 728 formed by the suction force.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An aortic leaflet crossing system comprising:
a guide catheter defining a grasper lumen and a guidewire lumen therein, the guide catheter having a proximal end defining a grasper lumen proximal opening and a guidewire lumen proximal opening, the guide catheter having a distal end defining a grasper lumen distal opening and a guidewire lumen distal opening;
a grasper configured to extend into the grasper lumen proximal opening and through the grasper lumen and out of the grasper lumen distal opening; and
a guidewire configured to extend into the guidewire lumen proximal opening and through the guidewire lumen and out of the guidewire lumen distal opening,
the grasper configured to grasp one or more aortic leaflets to form an opening across an aortic crossing, the opening configured to receive the guidewire extending out of the guidewire lumen distal opening.

2. The aortic leaflet crossing system of claim 1, wherein the grasper includes a first grabber having a first grabber distal portion and a second grabber having a second grabber distal portion, the first and second grabber distal portions configured to collectively grasp an aortic leaflet of the one or more aortic leaflets in an extension position.

3. The aortic leaflet crossing system of claim 2, wherein the first grabber distal portion includes one or more first grabbing features and/or the second grabber distal portion includes one or more second grabbing features.

4. The aortic leaflet crossing system of claim 3, wherein the one or more first grabbing features are formed on a first inner surface of the first grabber distal portion and/or the one or more second grabbing features are formed on a second inner surface of the second grabber portion.

5. The aortic leaflet crossing system of claim 1, wherein the grasper includes a distal end portion having a first portion, a second portion, and a bend connecting the first portion and the second portion, the second portion is configured to hook into an aortic leaflet of the one or more aortic leaflets in an extension position.

6. The aortic leaflet crossing system of claim 5, wherein the first portion biases outward a longitudinal axis of the guide catheter and the second portion biases inward toward the longitudinal axis in the extension position.

7. The aortic leaflet crossing system of claim 5, wherein the grasper is configured to retract to pull on the aortic leaflet of the one or more aortic leaflets to form the opening across the aortic crossing.

8. The aortic leaflet crossing system of claim 1, wherein the grasper includes a first grabber having a first grabber distal portion and a second grabber having a second grabber distal portion, the first and second grabber distal portions configured to grasp a first aortic leaflet and a second aortic leaflet, collectively, of the one or more aortic leaflets, in an extension position.

9. The aortic leaflet crossing system of claim 8, wherein the first grabber distal portion and the second grabber distal portion forms a clamp in the extension position.

10. The aortic leaflet crossing system of claim 8, wherein the first grabber distal portion hooks into the first aortic leaflet and the second grabber distal portion hooks into the second aortic leaflet in the extension position.

11. An aortic leaflet crossing system comprising:
a guide catheter defining a grasper lumen and a guidewire lumen therein, the guide catheter having a proximal end defining a grasper lumen proximal opening and a guidewire lumen proximal opening, the guide catheter having a distal end defining a grasper lumen distal opening and a guidewire lumen distal opening, the guide catheter extending along a guide catheter longitudinal axis, the grasper lumen extending along a grasper lumen longitudinal axis, and the guidewire lumen extending along a guidewire lumen longitudinal axis;
a grasper configured to extend into the grasper lumen proximal opening and through the grasper lumen and out of the grasper lumen distal opening; and
a guidewire configured to extend into the guidewire lumen proximal opening and through the guidewire lumen and out of the guidewire lumen distal opening,
the grasper configured to grasp one or more aortic leaflets to form an opening across an aortic crossing, the opening configured to receive the guidewire extending out of the guidewire lumen distal opening.

12. The aortic leaflet crossing system of claim 11, wherein the grasper lumen longitudinal axis and the guidewire lumen longitudinal axis are offset the guide catheter longitudinal axis.

13. The aortic leaflet crossing system of claim 11, wherein the guidewire lumen longitudinal axis and the guide catheter longitudinal axis are coextensive.

14. The aortic leaflet crossing system of claim 11, wherein the grasper lumen includes a first grasper lumen having a first grasper lumen longitudinal axis and a second grasper lumen having a second grasper lumen longitudinal axis, the first grasper lumen longitudinal axis and the second grasper lumen longitudinal axis are offset the guide catheter longitudinal axis.

15. The aortic leaflet crossing system of claim 14, wherein the first grasper lumen longitudinal axis, the second grasper lumen longitudinal axis, and the guide catheter longitudinal axis are coextensive along a plane.

16. An aortic leaflet crossing method comprising:
inserting a guide catheter defining a grasper lumen and a guidewire lumen therein into a patient's vasculature, the guide catheter having a proximal end defining a grasper lumen proximal opening and a guidewire lumen proximal opening, the guide catheter having a distal end defining a grasper lumen distal opening and a guidewire lumen distal opening, the grasper lumen carrying a grasper and the guidewire lumen carrying a guidewire;
delivering the guide catheter to an aorta;
advancing the grasper relative to the guide catheter out of the grasper lumen distal opening;
grasping one or more aortic leaflets with the grasper;
forming an opening across an aortic crossing with the grasper; and
advancing the guidewire relative to the guide catheter out of the guidewire lumen distal opening and through the opening across the aortic crossing.

17. The aortic leaflet crossing method of claim 16, wherein the forming step includes retracting the grasper when it is grasping the one or more aortic leaflets.

18. The aortic leaflet crossing method of claim 16, wherein the grasping step includes grasping a first aortic leaflet and a second aortic leaflet of the one or more aortic leaflets to form the opening at a cusp between the first aortic leaflet and the second aortic leaflet.

19. The aortic leaflet crossing method of claim 18, wherein the grasping step includes clamping the first aortic leaflet and the second aortic leaflet between the grasper.

20. The aortic leaflet crossing method of claim 16, wherein the grasping step includes hooking the first aortic leaflet and the second aortic leaflet.
